(19) United States
(12) Patent Application Publication
(10) Pub. No.: US 2013/0078701 A1
(43) Pub. Date: Mar. 28, 2013

(54) DEVICE COMPRISING A CONDUCTIVE SURFACE AND A CONDUCTIVE POLYMER FOR ADHESION OF CELLS AND TISSUE

(71) Applicants: Kristin Persson, Stockholm (SE); Roger Karlsson, Norrkoping (SE); Magnus Berggren, Vreta Kloster (SE); Peter Konradsson, Skanninge (SE); Edwin Jager, Linkoping (SE); Agneta Richter-Dahlfors, Saltsjo-Boo (SE); Karl Svennersten, Stockholm (SE)

(72) Inventors: Kristin Persson, Stockholm (SE); Roger Karlsson, Norrkoping (SE); Magnus Berggren, Vreta Kloster (SE); Peter Konradsson, Skanninge (SE); Edwin Jager, Linkoping (SE); Agneta Richter-Dahlfors, Saltsjo-Boo (SE); Karl Svennersten, Stockholm (SE)

(73) Assignee: OBOE IPR AB, Norrkoping (SE)

(21) Appl. No.: 13/639,595

(22) PCT Filed: Apr. 6, 2010
(86) PCT No.: PCT/EP10/54545

§ 371 Date: Dec. 5, 2012

(51) Int. Cl. C12N 13/00

(52) U.S. Cl. CPC C12N 13/00
USPC 435/173.1; 435/283.1

(57) ABSTRACT

The present disclosure relates to a device comprising a conductive substrate surface (19), at least one layer of a conductive polymer (14) deposited on the surface (19), a first electrolyte (13) arranged in contact with the conductive polymer layer, and a counter electrode (11), arranged in contact with the first electrolyte (13), such that a potential difference can be applied between the conductive substrate (15) and the counter electrode (11).

The conductive polymer (14), in a first state, before applying the potential difference, exhibits a first adhesive capacity, wherein the conductive polymer layer (14) is substantially attached to the conductive substrate surface (19). In a second state, subsequent to application of the potential difference, the conductive polymer (14), exhibits a second adhesive capacity, such that at least a portion of the conductive polymer layer (14) is substantially released from the conductive substrate surface (19).

### TECHNICAL FIELD

The present document relates to a device for electronic release of a conductive polymer from a surface, to a system for releasing cells from a surface of such a device, to a method using such a device and to a method of manufacturing such a device. The present document further relates to a conductive polymer.

### BACKGROUND

Today, adherent cells are typically detached by enzymatic or mechanical methods. Both methods may cause damage to cells as well as matrix and membrane bound proteins. When studying adherent cells and tissue cultured on solid support, e.g. on cell culture dishes and in flasks, there is a need to be able to release these cells for further propagation, subculture, or analysis. Therefore there is a need for an alternative cell release method that does not cleave proteins and damage cells. This is of importance in for example tissue engineering where cells are integrated into functional tissues such as the epidermis where membrane proteins achieving cell-cell contacts are absolutely necessary for the function of the tissue. The conservation of membrane proteins is of importance in many other cell biology applications as well. The urge for selective detachment and harvesting of cells and tissue structures is not only limited to living cells and tissues, also fixed cell and tissue specimens are of interest. In this case specific cells such as tumor cells could be isolated from a tissue specimen for genomic analysis.

M. Kim et al, Chem Comm 2009 discloses a heparin hydrogel which is attached to ITO electrodes. Cells are grown on the hydrogel. When applying a potential, the hydrogel with the cells are detached due to desorption of the silane groups holding the hydrogel in place. The hydrogel and the cells growing on it can be moved to another substrate for further growth. In the disclosed document, the hydrogel that carries the cells are not directly attached to the electrode below, but through a silane layer, and it is that layer that is responsible for the detachment. Cells do remain attached to a carrier substrate, namely the hydrogel, after delamination. The fact that the cells remain attached to the hydrogel after release may be a problem in some applications where free cells are preferred.

T. Okano et al, J biomed Mat Res 1993 discloses a recovery system for cultured cells using plasma-treated polystyrene dishes grafted with poly(N-isopropylacrylamide). Below 32° C. poly(N-isopropylacrylamide) has a fully expanded chain conformation resulting in a hydrophilic surface. Above 32° C. poly(N-isopropylacrylamide) collapses to a compact conformation resulting in a hydrophobic surface. In the disclosed document rat hepatocytes and bovine endothelial cells are cultured on the surfaces at 37° C. When the temperature is reduced below the polymer transition temperature both rat hepatocytes and bovine endothelial cells are detached from the poly(N-isopropylacrylamide) surface. Cells recovered by this method maintain substrate adhesivity, growth, and secretion activities nearly identical to those found in primary cultured cells in contrast to the compromised function found in cultured cells damaged by trypsinization. This technique is limited by the fact that it is temperature dependent. The temperature change could have effect on cellular processes, and may make it rather difficult to selectively detach specific cells since selective cooling of small areas is technically difficult.

W. Yeo et al, Langmuir 2006 discloses a self assembled monolayer, SAM, with an electroactive group tethering a peptide for cell adhesion. Cells will adhere to the monolayer through the peptide. The electroactive group can be cleaved when a potential is applied. This will lead to release of the part containing the peptide, taking with it any adhered cells. By using different electroactive groups, cell release can be achieved at different potentials. When the different electroactive groups are used on the same substrate, cells can be released at different times from one substrate. The SAMs can be patterned on gold using different methods, such as soft lithography. The manufacturing of the SAMs may be somewhat complicated and involves many steps of chemical synthesis. SAMs are generally anchored to gold surfaces, which are not ideal for microscopy studies as gold is not transparent.

Emmert-Buck et al, Science 1996 discloses a method for rapid one-step procurement of selected cell populations from a section of complex, heterogenous tissue under direct microscopic visualization. The method entails placing a thin transparent film over a tissue section, visualizing the tissue microscopically, and selectively adhering the cells of interest to the film with a fixed-position short-duration, focused pulse from an infra-red laser. Once the cells of interest are adhered to the film they can be placed directly into DNA, RNA, or enzyme buffer for further analysis. Transfer film activation can be accomplished with a variety of lasers. In the disclosed document a carbon dioxide laser is used. The method has good spatial resolution, but it is only applicable to dead cells and tissues, and it depends on advanced technical equipment.

The Cell Stripper™ product sheet discloses a non-enzymatic cell dissociation solution designed to detach adherent cells in culture without the risk of damage associated with trypsin solutions. The solution described in the disclosed document is a balanced salt solution with a mixture of chelators, which gently dislodges adherent cells in culture. This method does not allow any spatial selectivity of the cell detachment, and it does not discriminate between cell cell adhesions and cell substrate adhesions which makes it unsuitable for detachment of intact cell sheets.

Therefore there is a need for an alternative cell release method that does not cleave proteins and damage cells. This is of importance in for example tissue engineering where cells are dependent on functional membrane proteins for their cell to cell contacts. This is also of importance in many cell biology applications. There is also a need for a device and method for releasing cells from a surface of such a device which allows for a good spatial and temporal control of the release of cells and other objects.

### SUMMARY

It is an object of the present disclosure, to provide an improved device for detaching objects from a surface, in particular cells or tissue cultured on a surface which eliminates or alleviates at least some of the disadvantages of the prior art.

More specific objects include providing a device in which the detachment of cells can be spatially and temporally controlled in a selective manner.

The object is wholly or partially achieved by a device and a method according to the appended independent claims. Embodiments are set forth in the appended dependent claims, and in the following description and drawings.

According to a first aspect there is provided a device comprising: a conductive substrate surface, at least one layer of a conductive polymer deposited on the surface, a first electrolyte arranged in contact with the conductive polymer layer, and a counter electrode, arranged in contact with the first electrolyte, such that a potential difference can be applied between the conductive substrate and the counter electrode. the conductive polymer, in a first state, before applying the potential difference, exhibits a first adhesive capacity, wherein the conductive polymer layer is substantially attached to the conductive substrate surface. The conductive polymer. in a second state, subsequent to application of the potential difference, exhibits a second adhesive capacity, whereby at least a portion of the conductive polymer layer is substantially released from the conductive substrate surface.

The conductive polymer may be amphiphilic which means that it has the ability to be polar and water soluble and non-polar, i.e. soluble in organic solvents. This causes the polymer to be able to attach or adhere to non-polar surfaces, i.e. Orgacon® of the conductive substrate and remain there despite being surrounded by water. This means that the conductive polymer may have an adhesive capacity in a first state where it remains attached to the conductive substrate and may be able to change the adhesive capacity to a second state where it releases or delaminates completely from the conductive substrate, when a potential difference is applied across the material.

By “adhesive capacity” is meant the capacity or ability of the conductive polymer to remain attached or adhered to the conductive substrate where it is deposited.

By “substantially attached” is meant that when the conductive polymer has been deposited onto the conductive substrate, and when the material has been brought into contact with the electrolyte it still remains effectively fixated to the surface of the conductive substrate.

By “release” is meant that the conductive polymer effectively delaminates from the surface of the conductive substrate, such that there is substantially no conductive polymer which remains attached to the surface, this means that in the second state the adhesive capacity is close to zero. By “delaminate” is meant that the conductive polymer detaches from the conductive substrate. In the below the expression delaminate will be used to describe that the conductive polymer releases from the surface of the conductive substrate.

This can be affected through change in physical properties, reduction of inter/intra molecular forces, dissolving or disintegrating depending on the type of material. The conductive polymer stays attached to the substrate when no potential is applied, i.e. when the adhesive capacity of the conductive polymer is in a first state.

The device of the invention enables selective release or delamination with high spatial and temporal control of a polymer film from a conductive substrate. This is of primary importance for detachment of adherent cells cultured on a solid support e.g. cell culture dishes and flasks. Currently, adherent cells are typically detached by enzymatic or mechanical methods. Both methods may cause damage to cells as well as matrix and membrane bound proteins. This device allows for an alternative cell release method that does not cleave proteins and damage cells.

By this device detachment of for instance cells can be accomplished by release or delamination of the electroactive substrate from a surface of the conductive material. This leaves the detached cells fully intact and unharmed. As precise control of detachment may be realised by delamination of the conductive polymer layer only occurring when applying a potential, i.e. when the adhesive capacity changes from the first to the second state, and the rate of the delamination can be monitored by the magnitude of the potential.

The invention provides a simple means of releasing a whole layer of cells, or other materials deposited on top of a conductive polymer. The released layer can be collected as a whole, with no remains of the conductive polymer it was previously attached to since the conductive polymer layer is delaminated and subsequently disintegrated or dissolved simply by applying a potential. This is particularly important for applications where no foreign material or substances may be in contact with the cells or tissue. For adherent cells this can be solved by washing after adhesion is accomplished. It is also possible to use a conductive polymer that is completely dissolved upon release or delamination from the surface of the conductive substrate.

The rate of release may be controllable by the magnitude of the potential difference applied.

The device may further comprise an additional material layer, arranged between the conductive polymer and the first electrolyte.

This additional layer must still allow for the electrolyte to be in contact with the conductive polymer layer.

The additional material layer may comprise cells or tissue.

This allows for a device wherein cells may be effectively detached from the surface of the conductive substrate since the cells are arranged onto the conductive polymer layer which may release from the conductive substrate when a potential difference is applied across the conductive polymer layer.

The device may further comprise a second electrolyte arranged between the conductive polymer layer and the additional material layer.

The second electrolyte may be a solid electrolyte layer.

By “solid electrolyte” is also meant e.g. gel electrolytes.

According to one embodiment of the first aspect the device may further comprise at least one device unit, said device unit may, in turn, comprise a layer of a conductive polymer deposited on a conductive substrate, a solid electrolyte layer arranged to be in contact with the conductive polymer, and a second conductive material layer arranged to be in contact with the *solid electrolyte layer.

According to one alternative, a stacked device arrangement may comprise a plurality of units separated by substantially insulating material layers.

This device allows for a stacked arrangement of the device, by applying a potential a portion of the stack may be removed, carrying the additional material layer with it, attached to the released part of the stack. A substantially insulating material is arranged to prevent conductance through the entire stacked device arrangement.

As the conductive polymer can delaminate from a substrate, stacks may be realized in which different electroactive layers can be addressed. For instance, several layers on top of each other can be detached in series which enable us to grow cell and tissues in several different environments, moving it from one to the next by detaching layers in sequence. When finally the last layer is addressed it enables full release of any additional tissue layer leaving it fully freestanding in the media.

The stacked device arrangement allows for repeatedly moving an intact cell or tissue culture to new culture systems. This requires a carrier layer that may be removed intact, taking the cells or tissue with it. By stacking several carrier layers on top of each other cells and tissue may be moved from one cell culture plate to another.

According to one alternative embodiment the first electrolyte may be a liquid electrolyte.

A liquid electrolyte may allow for an efficient removal of the cells detached from the surface of the conductive substrate when the conductive polymer has released or delaminated from that surface.

According to yet an alternative embodiment of the first aspect of the invention the conductive polymer layer and the conducting substrate layer may comprise a patterned arrangement, thereby forming at least two, as seen in a plane parallel to the conducting substrate layer, separate parts.

The conductive substrate may be arranged on a substantially insulating carrier material.

According to one alternative the substrate may comprise a chip having at least two individually addressable electrodes. The chip may comprise at least one inorganic or organic transistor.

This device allows for a potential to be applied to a selected part of the conductive substrate and thus for a selective release or delamination by patterning the conductive polymer and the underlying substrate to individually addressable parts or pads, and by addressing only the selected ones, local control of the delamination may be achieved.

This device allows for selectively detaching only the cells of interest down to a specificity of individual cells which may be of particular importance when working with heterogeneous cell cultures, for example co-cultures comprising different cell lines, primary cell preparations, and cell cultures where only a fraction of the cells is successfully transfected. The heterogenic nature of the cell cultures makes it difficult to do biochemical and molecular biology analysis. In addition cells may remain attached to portions of conductive polymer where no potential difference is applied.

This device also allows for a simple method of selectively removing and analysing specific structures or regions in a tissue specimen without a need to dissect out these special structures for further analysis as this dissection is time consuming and relies on experts with special training.

A patterned device also allows for rather small structures to be manufactured and the patterning into small structures enables selective detachment of individual cells or areas of cells.

The device according to the first aspect may further comprise a reference electrode arranged in contact with the first electrolyte.

This allows for a precise monitoring of the potential difference applied on the conductive polymer and hence for a precise monitoring of the rate of release of the conductive polymer layer.

According to one alternative embodiment the device may be arranged in a cell culture device, such as any one of a cell culture flasks or a Petri dish.

According to one alternative embodiment of the device according to the first aspect the counter electrode may comprise a second layer of conductive material and the first electrolyte may comprise a layer of solid electrolyte arranged to be in contact with the conductive polymer layer. According to this alternative embodiment the conductive substrate layer may be deposited on a first object, and the second layer of conductive material may be arranged between the solid electrolyte and a second object.

By this arrangement it is possible to apply a potential difference between the conductive support layer and the counter electrode or second layer of conductive material and thereby causing the first object to be removed from the second object or vice versa, by the release or delamination of the conductive polymer. In addition to biological problems, this technology may also serve a need to delaminate non-biological and larger objects, and function as an electronic glue for packages, envelopes etc., where release may be performed on demand by applying an electronic signal.

The conducting substrate layer may comprise any one of indium tin oxide, gold or conductive polymers, such as polypyrrole and PEDOT and combinations thereof.

The conductive polymer may include any one of poly(3,4-ethylenedioxythiophene), poly(pyrrole), polyanilines, polythiophenes, or polymer blends thereof.

The conductive polymer may be poly(4-(2,3-dihydrothieno [3,4-b]-[1,4]dioxin-2-yl-methoxy)-butane-1-sulfonic acid.

Poly(4-(2,3-dihydrothieno [3,4-b]-[1,4]dioxin-2-yl-methoxy)-butane-1-sulfonic acid (PEDOT-S) is a material with substantial degree of self-doping, which means that the anionic functionalities are, used intramoleculary as dopants for the oxidized polymer. Apart from self-doping the polymer also contains free dopant ions as charge-balancing counter ions from the polymerization process. The unique combination of self-doping and free dopants enable full water solubility, but also, creates an amphiphilic polymer with the possibility of adhesion to substrates and/or surfaces of different polarities.

The high conductivity of PEDOT-S together with results from elementary analyses and XPS measurements indicates a high doping level of PEDOT-S. The possibility of introducing more charges on the PEDOT-S backbone with a low and controlled voltage potential will result in less rotational freedom of the polymer chains and an volume expansion due to absorbance of counter ions, neutralizing the excess charges. When the PEDOT-S film is adhered to the conductive substrate, the amphiphilic nature of the polymer results in attractive forces between the layers. Once a potential is applied in contact solution, PEDOT-S delaminates from the substrate by the volume expansion due to the injected charges.

The liquid electrolyte may comprise any one of aqueous sodium chloride, phosphate buffered saline, cell media or blood.

According to a second aspect there is provided a system comprising a release device comprising: a conductive substrate surface, at least one layer of a conductive polymer deposited on the surface, a first electrolyte arranged in contact with the conductive polymer layer, and a counter electrode, arranged in contact with the first electrolyte, such that a potential difference can be applied between the conductive substrate and the counter electrode; and a group of cells or a tissue portion, grown on the conductive polymer. The conductive polymer, in a first state, before applying the potential difference, exhibits a first adhesive capacity, wherein the conductive polymer layer is completely attached to the conductive substrate surface; and wherein the conductive polymer, in a second state, subsequent to application of the potential difference, exhibits a second adhesive capacity, wherein at least a portion of the conductive polymer layer and thus the cells or tissue portion is released from the conductive substrate surface.

This system allows for cells or tissue to be effectively detached from the surface since the conductive polymer onto which they may be arranged is able to release or completely delaminate from the surface of the conductive substrate as described above for the first aspect of the invention.

According to a third aspect there is provided a use of a device according to the first aspect and a system according to the second aspect for releasing cells or tissue from a surface of the device, by applying a potential difference to the device such that the conductive polymer layer is released from the surface of the conductive substrate, and thus the cells or tissue is detached from the surface.

According to a fourth aspect there is provided a method for releasing a material layer from a surface. The method comprises providing a conductive polymer surface, the conductive polymer surface being arranged on a conductive substrate and presenting a first adhesive state, wherein the conductive polymer remains completely attached to the conducting substrate, providing the material layer on the conductive polymer surface, providing a first electrolyte in contact with the conductive polymer layer, and applying a potential difference to the conductive polymer layer, such that the conductive polymer assumes a second adhesive state, wherein at least a portion of the conductive polymer layer, and thus the material layer, is released from the conductive substrate.

By arranging the additional material layer in contact with the conductive polymer, which upon applying a potential difference releases from the surface of the conductive substrate, the additional material layer may also be caused to detach from the surface.

According to one alternative embodiment of the fourth aspect the conductive polymer and the conductive substrate are patterned into, as seen in a plane parallel to the conductive substrate, at least two isolated parts, the method may comprise applying a potential difference over at least one selected part of the conductive polymer, whereby the selected part of the conductive polymer releases from the conducting substrate.

According to one embodiment the additional material layer may comprise cells or tissue.

This method allows for selective isolation of specific structures on a tissue specimen and may be accomplished by selective release or delamination of the areas of interest and subsequent harvesting of the specific structures, e.g. with an adhesive tape.

The method further allows for organised co-cultures to be accomplished using the method in a two step process. The first step includes selective detachment of some areas of the first cell population leaving these areas available for adhesion of a second cell population. The second step involves the seeding of the second cell population so they may occupy the empty areas. These steps may be repeated to add cell type 3, 4, etc. That is, parts of the first and/or second cell population may be released followed by seeding cell population 3.

According to a fifth aspect there is provided a method of manufacturing a device according to the first aspect. The method comprises depositing a conductive polymer layer on a conducting substrate layer; and arranging an electrolyte to be in contact with the conductive polymer layer, and arranging a counter electrode in contact with the electrolyte.

The method of manufacturing a device according to the fifth aspect may further comprise arranging the conducting substrate by a patterning procedure into at least two, as seen in a plane parallel to the conductive substrate layer, isolated parts; and depositing a conductive polymer on the isolated parts of the conducting substrate.

According to one alternative the conductive polymer may be deposited on the conductive substrate by any one of spin coating, drop casting or bar coating.

The conductive polymer may be patterned by lifting off the conductive polymer from the pre-patterned conducting substrate, the conductive polymer may, as an alternative, be etched to correspond to the isolated portions of the conducting substrate which may have been patterned by photolithographic techniques before etching. The conductive polymer and the conducting substrate may also alternatively be etched simultaneously.

According to a sixth aspect, there is provided a method for growing cells or tissue. The method comprises providing a conductive polymer surface having a predetermined shape, the conductive polymer surface being arranged on a conductive substrate and presenting a first adhesive state, wherein the conductive polymer remains completely attached to the conducting substrate, growing cells or tissue on the conductive polymer surface, providing a first electrolyte in contact with the conductive polymer surface, and applying a potential difference to the conductive polymer layer, such that the conductive polymer assumes a second adhesive state, whereby at least a portion of the conductive polymer layer, is released from the conductive substrate.

The predetermined shape may be substantially planar. In the alternative, the predetermined shape may be non-planar. For example, the predetermined shape is substantially hemispherical, a segment of a sphere, a segment of an ellipsoid, a segment of an oblate spheroid, a segment of a prolate spheroid or a catenoid shape.

By this method it may be possible to grow cells, to assume a predetermined shape or size before e.g. transplanted or grafted into or onto the human or animal body. The cells may be for instance epithelial cells or corneal epithelium, thus allowing for skin to be grown on the polymer surface and subsequently released for transplanting or grafting, and having obtained the right size already when grown on the polymer surface. The method may further allow for a new cornea to be grown directly onto the polymer surface, having the right shape and then released as a whole for direct transplanting into the patients eye.

According to a seventh aspect, there is provided a conductive polymer having the general structural formula:

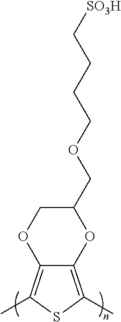

The conductive polymer may be poly(4-(2,3-dihydrothieno [3,4-b]-[1,4]dioxin-2-yl-methoxy)-butane-1-sulfonic acid.

According to one embodiment of the sixth aspect an adhesive capacity of the conductive polymer may be alterable in response to application of a potential difference over the conductive polymer, from a first state, wherein the conductive polymer substantially attached to a conductive substrate surface, to a second state, wherein the conductive polymer is substantially released from the conductive substrate surface.

### BRIEF DESCRIPTION OF THE DRAWINGS

Embodiments of the present solution will now be described, by way of example, with reference to the accompanying schematic drawings.

FIG. 1a-c schematically illustrate a side view of a device for delamination of a conductive polymer layer, with 1b showing the delamination and 1c including a reference electrode.

FIGS. 2a-2b schematically illustrates a side view of a device for delamination of a conductive polymer layer in a cell culture device.

FIGS. 3a-3d schematically illustrate a side view of a device for detachment of cells grown on a solid surface through release of a conductive polymer layer, with 3b showing the delamination and 3c the dissolving of the conductive polymer layer, and 3d including a reference electrode.

FIG. 4 schematically illustrates a side view of a device for detachment of cells from a solid surface in a cell culture device.

FIGS. 5a-5c schematically illustrate a side view of a device for detachment of cells and an additional layer of material through release of a conductive polymer layer, with 5b showing the dissolving conductive polymer layer and 5c including a reference electrode.

FIG. 6 schematically illustrates a side view of a device for detachment of cells and an additional layer of material placed in a cell culture dish.

FIG. 7 schematically illustrates a side view of a device for sequential detachment through release or delamination of a conductive polymer in a cell culture device.

FIGS. 8a-8b schematically illustrate a side view of a device for separating two objects from each other.

FIGS. 9a-9c schematically illustrate a side view of patterning of a conductive polymer layer, enabling a device for selective delamination of the conductive polymer layer.

FIGS. 10a-10b schematically illustrate a side view of a device for selective detachment of cells.

FIGS. 11a-11b schematically illustrate a top view and side view of the patterning of a conductive polymer layer into a matrix structure on a conducting substrate.

FIGS. 12a-12b schematically illustrate a side view and top view of the patterning of a conductive polymer layer into a matrix structure placed on a chip for individual addressing.

FIG. 13 illustrates the time of release or delamination (release time) at different magnitude of applied potentials.

FIG. 14 illustrates the time of release or delamination (release time) after soaking the conductive polymer for various times.

FIGS. 15a-15b illustrates selective release of one pixel in a matrix.

### DESCRIPTION OF EMBODIMENTS

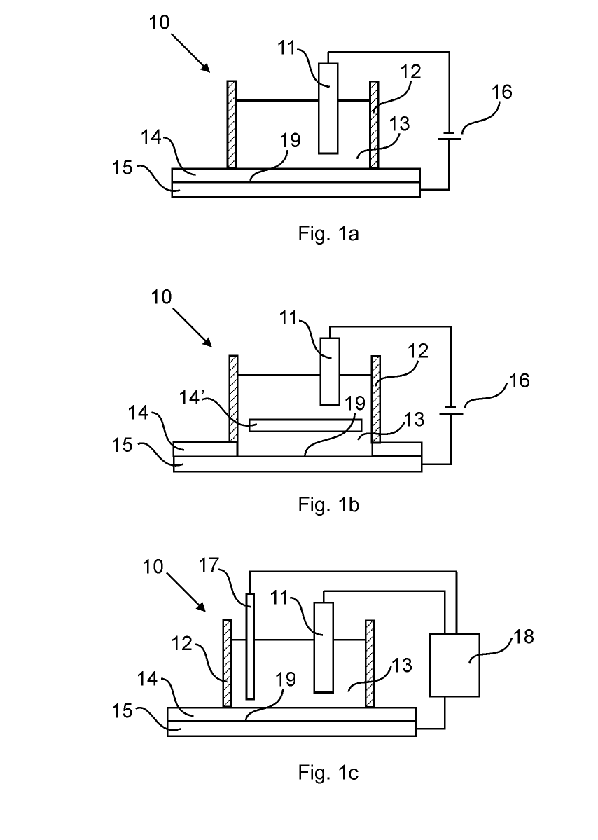

FIG. 1a illustrate a cross section of a device 10 comprising a layer of a conductive polymer 14 deposited on a conducting substrate 15.

The conductive polymer or electroactive material is placed in contact with a first electrolyte 13, in contact with which is also placed a counter electrode 11. A container 12 may be used for the first electrolyte 13, and the conductive polymer 14 and counter electrode 11 may be placed within the electrolyte. The container 12 may be of another shape than what is shown in FIG. 1a, for example the substrate 15 with the conductive polymer layer 14 could be placed along the edges of a container in a vertical fashion instead, opposite to the electrode 11. A potential may be applied between the conducting substrate 15 and electrode 11 by means of a power source 16, which could be a battery, a potentiostat or any other voltage generating device. The first electrolyte 13 may be any aqueous electrolyte including salts, buffers, cell media and biological fluids.

In FIG. 1a a first state of the adhesive capacity of the conductive polymer is shown. The conductive polymer may be completely attached to the conductive substrate, even if the conductive polymer is submerged in an electrolyte, due to the amphipilic character of the conductive polymer.

The first electrolyte 13 may also be a solid or gel electrolyte.

FIG. 1b illustrates the device 10 when a potential is applied between the substrate 15 and electrode 11, leading to delamination of the conductive polymer 14′ from the surface 19 of the conductive substrate 15. FIG. 1b thus shows a second state of the adhesive capacity of the conductive polymer 14′ in the conductive polymer 14′ is completely delaminated or released from the surface 19 of the conductive substrate 15. In the below this second state of the adhesive capacity will be generalised to that the conductive polymer layer 14 has release or delaminated from the surface 19 of the conductive substrate 15.

FIG. 1c illustrates the device 10 when a reference electrode 17 is placed in the electrolyte 13 for better control of the applied potential. To generate a voltage and potential difference, any device 18 compatible with a three-electrode set up can be used, including a potentiostat.

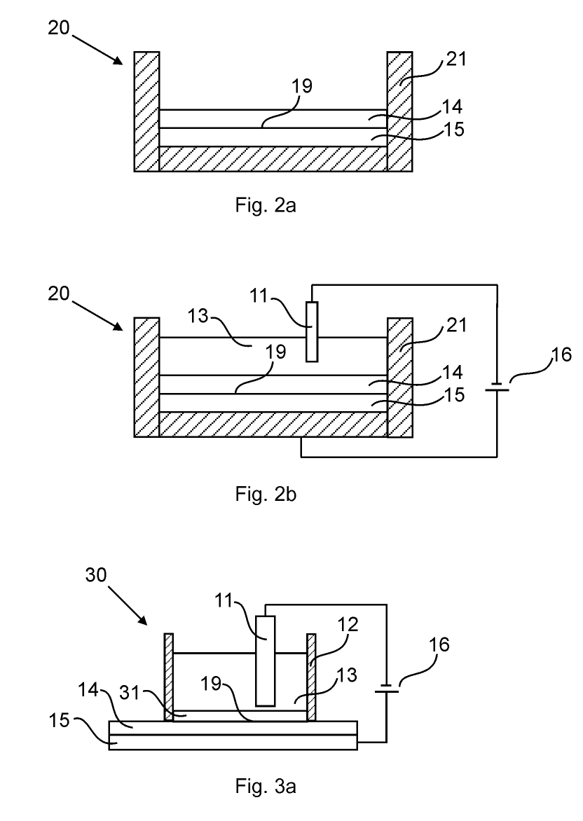

FIG. 2a illustrates a cross section of a device 20 wherein the conductive polymer 14, deposited on a surface 19 of a conducting substrate 15 is placed in a cell culture device 21, the walls of the cell culture device may then effectively constitute the walls 12 of the container.

FIG. 2b shows a device 20 placed in a cell culture device 21 and where a first electrolyte 13 has been arranged to be in contact with the conductive polymer 14. A counter electrode 11 has been arranged in the first electrolyte 13.

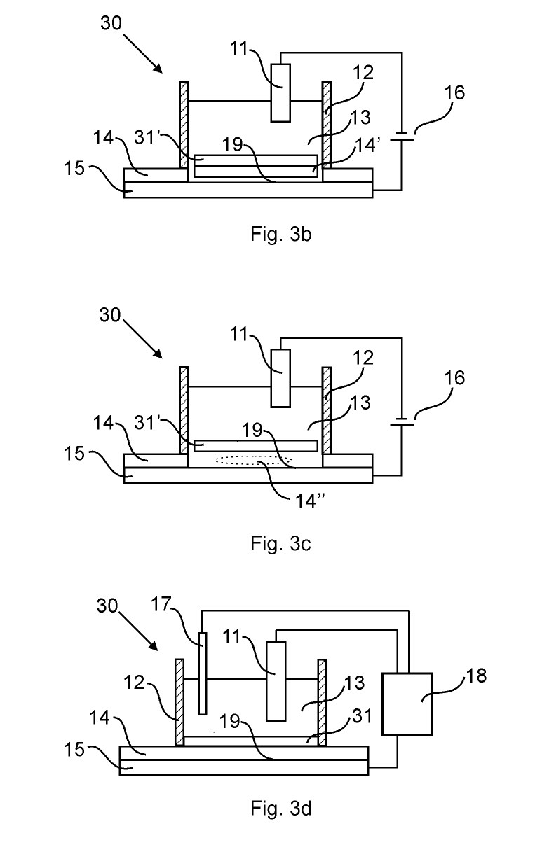

FIG. 3a illustrates a cross section of a device 30, differing from device 10 in that a layer of additional material 31 has been deposited on top of the conductive polymer layer 14, while still allowing for the conductive polymer layer to be in contact with the first electrolyte 13. The material 31 may be cells or tissue, but also other materials, forming for instance a thin film on the conductive polymer layer. The container 12 may act as a cell culture dish in the case of material 31 being cells. In case of cells, the first electrolyte 13 may alternatively be a cell culture medium.

FIG. 3b illustrates the device 30 when a potential is applied leading to delamination of the conductive polymer 14′, taking with it the deposited material, 31′ which then detaches from the surface 19.

FIG. 3c illustrates the device 30 when a potential has been applied and the conductive polymer 14″ has released or delaminated from the surface 19 of the conductive substrate 15, the conductive polymer subsequently dissolves or disintegrates, leaving the material 31′ intact and free standing.

FIG. 3d illustrates the device 30 when a reference electrode has been arranged similar to in FIG. 1c.

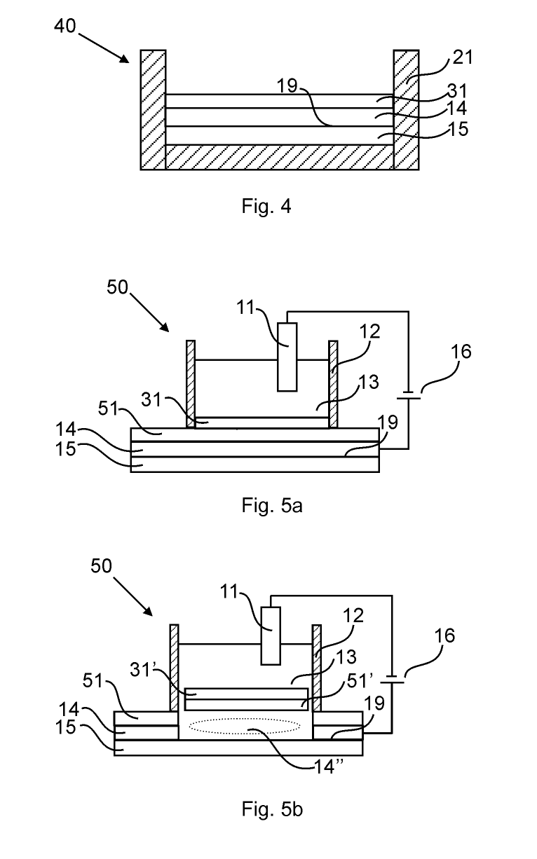

FIG. 4 illustrates a device 40 similar to device 20 but with an additional layer 31 which may be cells, tissue or another material. It should be clear that when adding first electrolyte 13, a counter electrode 11 and applying a potential between the counter electrode and the conducting substrate, the device may work as the one described in FIG. 2b.

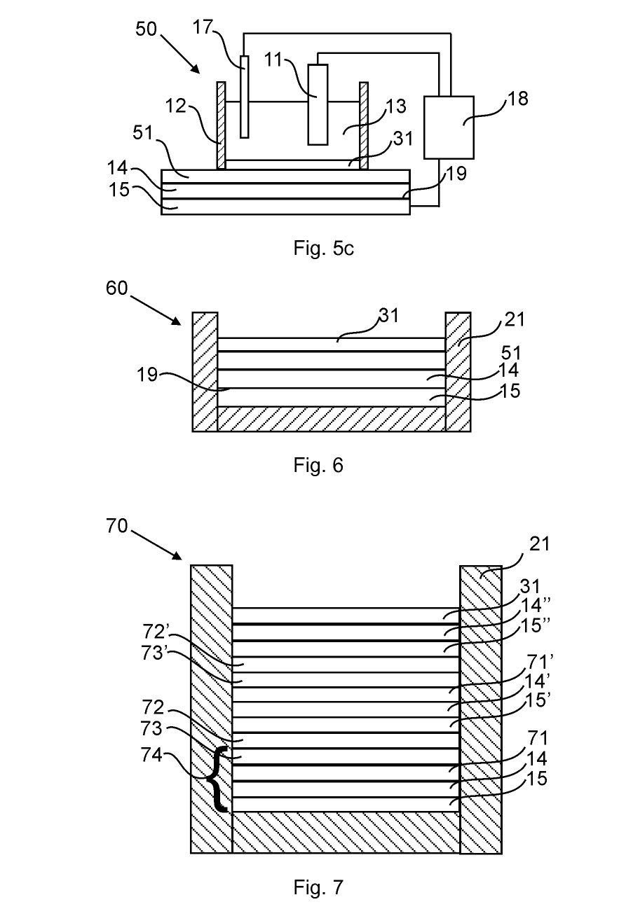

FIG. 5a illustrates a device 50 similar to device 30 but including a second additional material layer 51 placed in between the conductive polymer 14 and the additional material layer 31. The second additional material layer 51 may be a gel, a layer of proteins or a solid electrolyte. The second additional material layer 51 needs to be permeable to ions and other molecules in order to enable the conductive polymer 14 to undergo electrochemical reactions, i.e. allow for the conductive polymer 14 to be able to be in contact with the first electrolyte 13.

FIG. 5b illustrates the device 50 when a potential is applied, leading to release or delamination and subsequent dissolving or disintegration of the conductive polymer 14″, also leading to detachment of the layer 51′ and the layer 31′.

FIG. 5c illustrate the device 50 when a reference electrode has been arranged similar to the device in FIG. 1c.

FIG. 6 illustrates a device 60 similar to device 40 but with an additional layer 51 which may be a gel, a layer of proteins or a solid electrolyte. It should be understood that device 60 as shown in FIG. 6 may also comprise a first electrolyte 13 and a counter electrode as shown in FIG. 2b and that by applying a potential between the counter electrode and the conducting substrate, the device may work as the one described in FIGS. 5a-5c.

FIG. 7 illustrates a stacked device arrangement 70, that may comprise a plurality of device units 74 separated by an insulating layer 72.

A device unit 74 may comprise a layer of a conductive polymer 14 deposited on a conductive substrate 15, a solid electrolyte layer 71 arranged to be in contact with the conductive polymer 15, and a second conductive material layer 73 arranged to be in contact with the solid electrolyte layer 71. The second conductive material thus effectively may act as a counter electrode within the unit 74.

On top of the uppermost conductive polymer layer 14″ a layer of additional material 31, which alternatively may be cells may be arranged.

The stacked device arrangement 70 may alternatively be placed in a cell culture device 21.

A first electrolyte 13 may be arranged to be in contact with the uppermost layer of conductive polymer 14″. The first electrolyte may alternatively be a liquid electrolyte or a solid electrolyte.

A counter electrode 11 may be arranged to be in contact with the liquid first electrolyte 13. Alternatively a layer of a second conductive material is arranged to be in contact with a solid layer of a first electrolyte.

When applying a potential between one of the first conducting layers 15 and one of the second conducting layers 73, separated by a layer of conductive polymer 14 and the solid electrolyte layer 71, the conductive polymer layer 14 will delaminate. The layers in the stack above the conductive polymer layer 14, may be collected and moved to e.g. another cell culture device. By stacking more conductive polymer layers on top of each other than shown in the illustration, several subsequent delaminations may be performed, moving the top layer 31 from one media to another. Finally, the top most conducting layer 15″ can be addressed, leading the conductive polymer layer 14″ on top of it to delaminate from the surface 19 of the conducting material 15″.

The insulating layers may be provided to prevent electrical conductance through the entire stacked device arrangement.

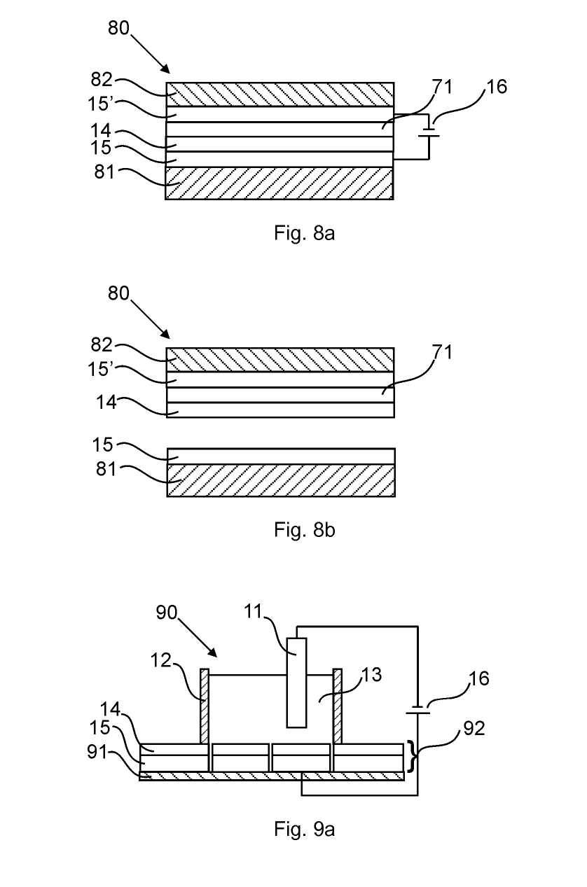

FIG. 8a illustrates a device 80 working as electronic glue, disrupting the interface between two solid objects 81 and 83, which may, but does not have to, be made of the same material. As an example, paper may be the solid object mentioned. In another example, the solid object could be parts of the device 10, 30 or 50 described above. A first and second conductive substrate material layer 15, 15′ may be placed on both solid objects, and the conductive polymer 14 may be deposited on one of the substrate material layers 15, 15′ The conductive materials used may be different, for example gold on one side and a conductive polymer on the other. Between the conductive polymer 14 and the second conductive material 15′, a solid electrolyte 71 may be placed. The shapes of the objects may be of a large variety, not limited to the ones shown in the figure. It is also possible to place more than two solid objects on top of each other, separated by layers of conductive polymer, conducting substrate and solid electrolyte. In that case, the solid objects can be detached from each other one by one.

FIG. 8b illustrates the device 80 when a potential is applied. The two solid objects may be separated. The upper object 82 may be detached from the lower object 81 or vice versa. As shown in the illustration, the conductive polymer 14 remains attached to the solid electrolyte 71, it may also remain attached to the underlying conductive layer 15, or it may be divided between 71 and 15.

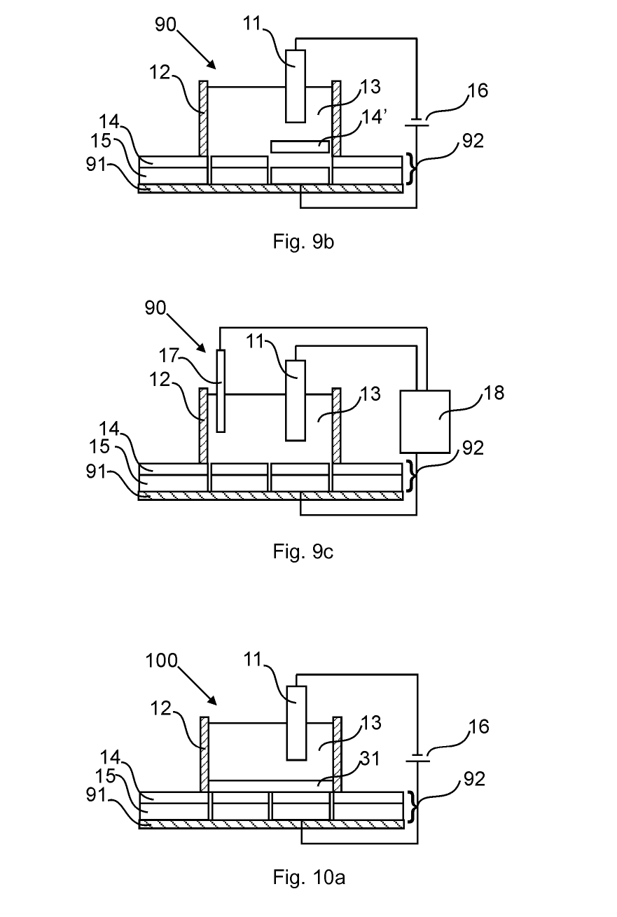

FIG. 9a illustrates a device 90 where the conductive substrate 15 has been patterned into a plurality of separate portion 92 as seen in a normal plane of the device. The conductive polymer 14 may be patterned into corresponding portions 92.

The device is similar to the device 10 as shown in FIG. 1a-1c, but the conducting substrate 15 here may be provided on an insulating carrier material 91. This carrier material may be plastic foil, glass or another insulating material on which the conductive material layer 15 may be deposited. As before, the conductive polymer 14 may be deposited on the conductive material layer 15.

Disruptions may be patterned on the conductive polymer 14 and the conductive material 15, creating isolated pads or portions 92 of conducting and conductive polymer.

Patterning may be achieved by photolithographic methods, dry etching, by screen printing and manually with aid of a scalpel or a similar device. By moving the connections to the voltage generator 16, individual addressing of the patterned pads or portions may be realized. It should be clear that the device 90 may be placed in a cell culture dish similar to the device 20 as shown in FIGS. 2a-2b.

FIG. 9b illustrates the delamination of one portion of the conductive polymer 14′, placed on the addressed part 92 of conducting substrate 15. The non-addressed portions of the conductive polymer 14 remain attached to the conducting material layer 15, i.e. remain in the first state of the adhesion capacity of the conductive polymer.

FIG. 9c illustrates device 90 with a reference electrode.

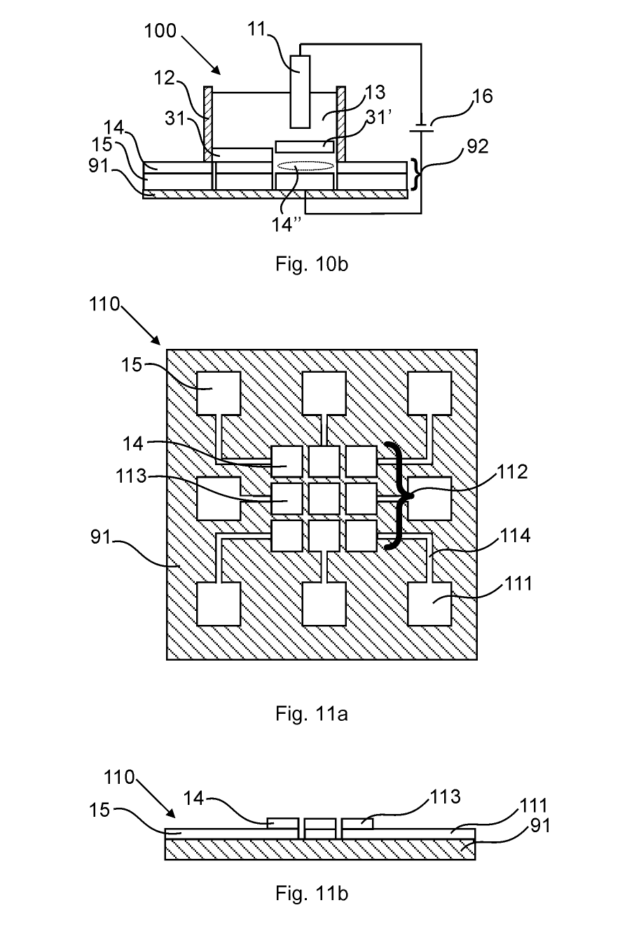

FIG. 10a illustrates a device 100, similar to 90 but with the addition of a layer of material 31. This layer may be cells, tissue or other materials. The electrolyte 13 may in the case of cells be cell medium. It should be clear that the device 100 may be placed in a cell culture dish similar to the device as shown in FIGS. 4a-4c.

FIG. 10b illustrates the device 100 when applying a potential to one of the parts or pads 92. This enables delamination and dissolving or disintegration of the conductive polymer 14″ on top of the addressed conductive material part. The additional material layer 31′ on top of the addressed portion of conductive polymer 14 may be detached, the rest of the additional material layer 31 may remain attached to the conductive polymer 14. If layer 31 is composed of cells, this may provide a method for selectively releasing only certain parts of a cell culture.

FIG. 11a illustrates a top view of a device 110 where a matrix has been patterned. A conducting substrate 15 has been deposited on an insulating carrier material 91. The conductive polymer 14 has been deposited on the conducting substrate 15. Both the conductive polymer 14 and the conductive substrate 15 has then been patterned into the matrix shape shown in FIG. 11a. The larger parts or pads 111 outside provide for individual addressing within the matrix 112 through conducting lines 114. The larger parts or pads 111 and the conducting lines 114 may consist of only the conducting substrate 15, or both the conductive polymer 14 and the conducting substrate 15. The smaller pads 113 in the matrix 112 consist of both the conductive polymer 14 and the conductive substrate 15. If patterning is done by means of screen-printing, conductive substrate layer 15 may be patterned first by other means such as photolithography and dry etching, followed by printing of the conductive polymer layer 14 in the desired areas. Alternatively, the conductive polymer layer 14 and the conductive substrate layer 15 may be patterned simultaneously by photolithography and dry etching. The matrix size is not limited to the 3×3 shown in the illustration, but should be thought of as a matrix of any number of rows and columns instead.

FIG. 11b illustrates a side view cross section of device 110. It should be clear that if a container is created using a material 12 forming the walls of the container and placed above the device, and electrolyte and a counter electrode is added, a device similar to the device 90 as shown in FIGS. 9a-9c may be formed.

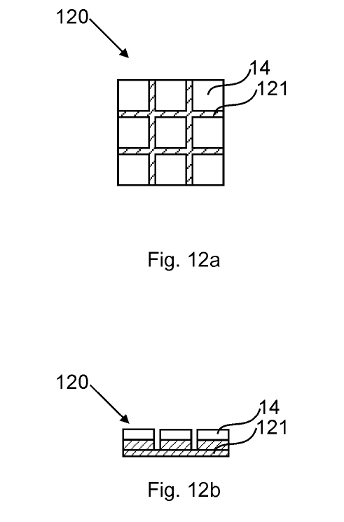

FIG. 12a illustrates a top view of a matrix structure on a chip, device 120. The conductive polymer 14 is placed on a chip 121, containing pads for individual addressing made of inorganic transistors and circuits. Disruptions are patterned in the conductive polymer to fit on the underlying pads in the chip. It should be clear that if a container is created using a material 12 and placed above the device, and electrolyte and a counter electrode is added, a device similar to the device as shown in FIGS. 8a-8c may be formed. The matrix size is not limited to the 3×3 shown in the illustration, but should be thought of as matrix having any number of rows and columns (n×n matrix) instead.

FIG. 12b illustrates a side view cross section of the device as shown in FIG. 12a.

In FIGS. 11a-11b and FIGS. 12a-12b it is clear that if a container is created on top of the matrix, an electrolyte, a counter electrode and an additional material layer 31 may be added, a device 90 similar to that shown in FIGS. 9a-9c may be formed. If the additional material layer 31 is cells, a matrix for cell release may be created. If using the chip 121 in the device 120, high precision and computerized control of cell release may be achieved.

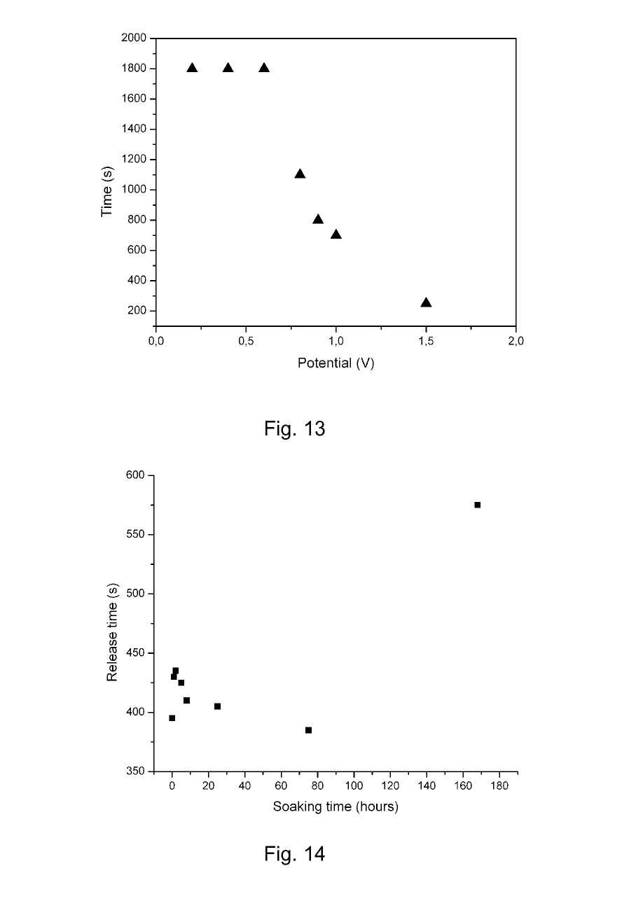

FIG. 13 illustrates that the magnitude of the applied potential influences the release time, meaning the time of release or delamination of the conductive polymer from the conducting substrate. Under the conditions applied here, a potential of at least 0.6 V (vs. Ag/AgCl) was needed for release or delamination.

FIG. 14 illustrates that soaking prior to applying a potential does not affect the time of release of the conductive polymer to any large extent.

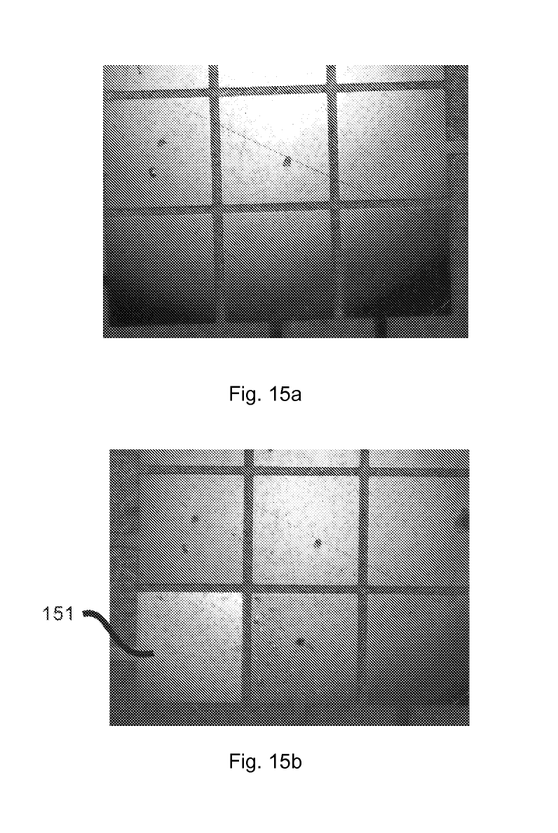

FIGS. 15a-15b illustrate a patterned substrate before (FIG. 15a) and after (FIG. 15b) applying a potential. After applying a potential, the lower left pixel has released or delaminated from the conductive substrate.

### EXAMPLES

In a first example, a conductive polymer (a PEDOT derivative) was bar coated on a conductive polymer substrate (in this case Orgacon™) and after drying it was placed in NaCl (aq) and a potential was applied between the conductive substrate and a platinum counter electrode, also placed in the electrolyte. A Ag/AgCl reference electrode was included to control the applied potential. The resulting device was similar to FIG. 1c. When a potential above 0.6 V was applied, the conductive polymer detached from the substrate through a process of charge injection and volume expansion (swelling) due to electrochemical reactions in the polymer chains, the swelling results in release or delamination from the substrate. If not applying a potential, the conductive polymer remained attached to the substrate. It was concluded that an applied potential can be used to induce release or delamination of a conductive polymer layer from a conductive substrate.

In a second example, the same set up as describe in the first example was employed, but the applied potential varied in magnitude between 0.1 and 1.5 V. The time of release or delamination was measured for the different potentials applied. It was discovered that the magnitude of the potential influences the time of release or delamination, a higher potential leading to a faster release or delamination. It was also discovered that there was a threshold potential, below which no delamination occurred. In the system used, this threshold was 0.6V. It was concluded that the rate of delamination can be controlled, and that the delamination seems to be controlled by electrochemical reactions only occurring above a certain threshold potential, depending on the conductive polymer used. The results are shown in FIG. 13.

In a third example, the same set up as describe in example 1 and 2 was employed. Before applying the potential, the substrate with the conductive polymer was soaked in the electrolyte, NaCl (aq) for up to one week. After soaking, a potential was applied and the release time was measured. It was discovered that release occurred even after soaking for up to one week (the longest time tested). A small increase in the release time was found, and it was concluded that pre-soaking in electrolyte does not affect the release times to a large extent. The result is shown in FIG. 14.

In a 4^{th} example, a conductive polymer (a PEDOT-derivative) was barcoated on a conducting substrate (Orgacon™) and after drying a ring of PDMS was glued on top of it, creating a cell culture device, similar to the one shown in FIGS. 3 and 4. Cells in serum containing media were cultured for 24 hours on the substrate. After 24 hours the amount of lactate dehydrogenase (LDH) was measured in the media, to investigate the cell viability on the conductive polymer. The measurement of LDH did not show an increased release due to culture on the conductive polymer.

In a 5^{th} example, a conductive polymer (a PEDOT-derivative) was barcoated on a conducting substrate (Orgacon™) and after drying a ring of PDMS was glued on top of it, creating a cell culture device, similar to the one shown in FIGS. 3 and 4. Cells in serum containing media were cultured for 24 hours on the substrate. Before a potential was applied between the conductive polymer substrate and a platinum counter electrode the cell media was replaced by a buffered salt solution. After application of the potential, samples were first taken to measure the amount of LDH in the solution and then the surface could be detached by repetitive pipetting and the cells harvested. The buffered salt solution containing cells and PEDOT fragments was transferred to a centrifuge tube. After centrifugation, cells and PEDOT form a pellet and the supernatant can be removed. The pellet is then reconstituted in cell culture media and the cells and PEDOT fragments can be reseeded on a cell culture surface. After incubation in a cell culture incubator cells adhere to the cell culture surface and PEDOT fragments can be removed by washing with fresh cell culture media. Results showed that the measured amount of LDH did not show any increase due to the applied potential. Cells grown on top of the conductive polymer were released when the layer delaminated. Reseeded cells could adhere to a new cell culture surface. After washing more than 90% of the PEDOT fragments could be removed from the culture.

A 6^{th} example refers to patterning and a device similar to those shown in FIGS. 9 and 11. Two different patterning methods were employed. The first method was based on lift-off, where photolithography was used to pattern the desired shapes on a sheet of Orgacon™. The conductive polymer was subsequently bar coated on top of the pattern. When washing with acetone, the pattern is dissolved, taking with it the conductive polymer on top, leaving conductive polymer in an inverted pattern. The second method was based on dry etching. In this case the conductive polymer was barcoated on Orgacon™, and both those layers were then patterned with photolithography. After that, a pattern was made with dry etching, and after removal of the patterned photoresist it resulted in conducting shapes separated by non conducting areas (as in FIG. 11).

In a 7^{th} example, patterned areas in a matrix form as in FIG. 11, were used to selectively delaminate the pixels one by one. A PDMS ring was glued on top of the matrix creating a device similar to FIG. 9, and the resulting container was filled with NaCl (aq). A gold wire was used as counter electrode and was placed in the electrolyte. When applying a potential between one of the outer pads and the counter electrode, the corresponding pixel of conductive polymer delaminated. The other pixels were not affected by the applied potential. The result is shown in FIGS. 15a-15b, where in FIG. 15b, the lower left pixel has been delaminated, i.e. has released from the conductive substrate.

1. A device comprising:
a conductive substrate surface,
at least one layer of a conductive polymer,
a first electrolyte arranged in contact with the conductive polymer layer, and
a counter electrode, arranged in contact with the first electrolyte, such that a potential difference can be applied between the conductive substrate and the counter electrode, wherein
the conductive polymer, in a first state, before applying the potential difference, exhibits a first adhesive capacity, wherein the conductive polymer layer is substantially attached to the conductive substrate surface; and
the conductive polymer, in a second state, subsequent to application of the potential difference, exhibits a second adhesive capacity, whereby at least a portion of the conductive polymer layer is substantially released from the conductive substrate surface.

2. The device as claimed in claim 1, wherein a rate of release is controllable by a magnitude of the potential difference applied.

3. The device as claimed in claim 1, wherein the device further comprises an additional material layer, arranged between the conductive polymer layer and the first electrolyte.

4. The device as claimed in claim 3, wherein the additional material comprises cells or tissue.

5. The device as claimed in claim 4, wherein the device further comprises a second electrolyte arranged between the conductive polymer layer and the additional material layer.

6. The device as claimed in claim 5, wherein the second electrolyte is a solid electrolyte layer.

7. (canceled)

8. (canceled)

9. (canceled)

10. The device as claimed in claim 1, wherein the conductive polymer layer and the conductive substrate layer comprises a patterned arrangement, thereby forming at least two, as seen in a plane parallel to the conductive substrate layer, separate parts.

11. (canceled)

12. The device as claimed in claim 1, wherein the conductive substrate comprises a chip having at least two individually addressable electrodes.

13. The device as claimed in claim 12, wherein the chip comprises at least one inorganic or organic transistor.

14. (canceled)

15. The device as claimed in claim 1, wherein the device is arranged in a cell culture device.

16. The device as claimed in claim 1, wherein the counter electrode comprises a second layer of conductive material and wherein the first electrolyte comprises a layer of solid electrolyte arranged in contact with the conductive polymer layer.

17. The device as claimed in claim 16, wherein the conductive substrate layer is deposited on a first object, and wherein the second layer of conductive material is arranged between the solid electrolyte and a second object.

18. The device as claimed in claim 1, wherein the conductive substrate layer comprises any one of indium tin oxide, gold or conductive polymers, and combinations thereof.

19. The device as claimed in claim 1, wherein the conductive polymer layer includes any one of poly(3,4-ethylenedioxythiophene), poly(pyrrole), polyanilines, polythiophenes, or polymer blends thereof.

20. The device as claimed in claim 1, wherein the conductive polymer is poly(4-(2,3-dihydrothieno [3,4-b]-[1,4]dioxin-2-yl-methoxy)-butane-1-sulfonic acid.

21. (canceled)

22. A system comprising:
a release device comprising: a conductive substrate surface, at least one layer of a conductive polymer deposited on the surface, a first electrolyte arranged in contact with the conductive polymer layer, arranged in contact with the first electrolyte, such that a potential difference can be applied between the conductive substrate and the counter electrode; and
a group of cells or a tissue portion, grown on the conductive polymer,
wherein the conductive polymer, in a first state, before applying the potential difference, exhibits a first adhesive capacity, wherein the conductive polymer layer is completely attached to the conductive substrate surface; and
wherein the conductive polymer in a second state, subsequent to application of the potential difference, exhibits a second adhesive capacity, whereby at least a portion of the conductive polymer layer and thus the group of cells or tissue portion is released from the conductive substrate surface.

23. (canceled)

24. A method for releasing a material layer from a surface, the method comprising:
providing a conductive polymer surface, the conductive polymer surface being arranged on a conductive substrate and presenting a first adhesive state, wherein the conductive polymer remains completely attached to the conducting substrate,
providing the material layer on the conductive polymer surface,
providing a first electrolyte in contact with the conductive polymer layer, and
applying a potential difference to the conductive polymer layer, such that the conductive polymer assumes a second adhesive state, wherein at least a portion of the conductive polymer layer and thus the material layer, is released from the conductive substrate.

25. The method as claimed in claim 24, wherein the conductive polymer and the conductive substrate are patterned into, as seen in a plane parallel to the conductive substrate, at least two isolated parts, the method further comprises applying a potential difference over at least one selected part of the conductive polymer layer, such that the selected part of the conductive polymer layer releases from the conducting substrate.

26. The method as claimed in claim 24, wherein the additional material layer comprises cells or tissue.

27. (canceled)

28. (canceled)

29. (canceled)

30. A method for growing cells or tissue, the method comprising:
providing a conductive polymer surface having a predetermined shape, the conductive polymer surface being arranged on a conductive substrate and presenting a first adhesive state, wherein the conductive polymer remains completely attached to the conducting substrate,
growing cells or tissue on the conductive polymer surface,
providing a first electrolyte in contact with the conductive polymer surface, and
applying a potential difference to the conductive polymer layer, such that the conductive polymer assumes a second adhesive state, whereby at least a portion of the conductive polymer layer, is released from the conductive substrate.

31. The method as claimed in claim 30, wherein the predetermined shape is substantially planar.

32. The method as claimed in claim 30, wherein the predetermined shape is non-planar.

33. (canceled)

34. (canceled)

35. (canceled)

36. (canceled)
